(19) United States
(12) Patent Application Publication
(10) Pub. No.: US 2013/0104905 A1
(43) Pub. Date: May 2, 2013

(54) CONDOM HAVING A GEL STORAGE PAD CAP

(71) Applicant: Il Chul Shin, Seongnam Si (KR)

(72) Inventor: Il Chul Shin, Seongnam Si (KR)

(21) Appl. No.: 13/805,665

(22) PCT Filed: Jul. 1, 2010
(86) PCT No.: PCT/KR2010/004296

§ 371 Date: Dec. 20, 2012

(30) Foreign Application Priority Data
Jun. 21, 2010 (KR) 10-2010-0058729

(51) Int. Cl. A61F 6/04

(52) U.S. Cl. CPC A61F 6/04
USPC 128/844

(57) ABSTRACT

Disclosed therein is a condom including a gel pad cap detachably fit to an inlet ring of the condom to thereby stop an inlet of the condom in a state where the condom is rolled, the gel pad cap containing a gel therein. The gel pad cap (20) includes a pad body (21) and a pad lid (23) which are separably joined, and the pad body (21) stores a gel therein and has an inlet (22) formed at a front flank of the pad body (21) joined to the pad lid (23) for discharging the gel to the outside.

### TECHNICAL FIELD

The present invention relates to a condom, and more particularly, to a condom, which is carried together with a lubricant to thereby increase convenience in keeping and carrying and enhance practicality, and which prevents that sex medicines for men coated on the inner surface of the condom and the lubricant coated on the outer surface of the condom are mixed.

### BACKGROUND ART

In general, a condom is made of a soft rubber material, is of a tubular form in such a way as to be covered on the male genital organ, and is distributed in a state where it is sealably packed with a vinyl bag.

Such a condom has been typically used to prevent a woman's pregnancy, but recently, people have advised to use condoms as aids to prevent various diseases, such as AIDS, venereal diseases, and so on. So, various kinds of condoms suitable for consumers' tastes have been manufactured, and now, they become indispensable necessaries in the modern society. In order to effectively use the condom, a user sometimes applies a lubricant on the outer surface of the condom. Such a lubricant is sold on the market in a state where it is put in a container, such as a vinyl container or a plastic container, because it is not disposable but is usable dozens of times, and hence, the user takes out and uses the lubricant a little if necessary. Various kinds of fragrances may be added to the lubricant according to consumers' tastes. Accordingly, the typical condom has a disadvantage in that the user has to carry the condom and the lubricant separately to apply the lubricant to the condom.

In the meantime, some of the condoms have a sex medicine for men coated on the inner surface thereof in order to treat premature ejaculation or erectile dysfunction. In this instance, the lubricant and the condom are put in one packing container, and hence, the sex medicine for men and the lubricant are mixed together and cannot show their own functions.

As described above, in the case that the condom and the lubricant are separately kept in safety or separately carried or in the case that the condom and the lubricant are kept in one packing container, the condom cannot show its function because the lubricant is stained on the inner surface of the condom and the condom is easily taken off, or because the sex medicine for men coated on the inner surface of the condom and the lubricant are mixed together.

### DISCLOSURE

### [Technical Problem]

Accordingly, the present invention has been made in an effort to solve the above-mentioned problems occurring in the prior arts, and it is an object of the present invention to provide a condom with a gel pad cap, which has a gel pad cap joined to the condom to thereby allow a user to easily carry and keep the condom and a gel (lubricant).

### [Technical Solution]

To achieve the above objects, the present invention provides a condom with a gel pad cap including: a gel pad cap detachably fit to an inlet ring of the condom to thereby stop an inlet of the condom in a state where the condom is rolled, the gel pad cap containing a gel therein.

It is preferable that the gel pad cap comprises a ring insertion groove formed on a flank of the outer face thereof so that the inlet ring of the condom is closely fit to the gel pad cap. Moreover, it is preferable that the gel pad cap and the inlet ring of the condom are closely fit with each other when the inlet ring 11 of the condom presses the outer surface of the gel pad cap 20 by a compressive force through an elastic force of the inlet ring, so that the gel pad cap is detachably fit to the inlet ring of the condom. For this, in the rolled state of the condom, the inlet ring of the condom is in a circular shape and the gel pad cap is in an oval shape or a polygonal shape. Alternatively, in the rolled state of the condom, the inlet ring of the condom is in a circular shape and the gel pad cap is also in a circular shape.

It is preferable that the gel pad cap is in a flat plate form.

It is preferable that the gel pad cap comprises a pad body and a pad lid which are separably joined, and the pad body stores a gel therein and has an inlet formed at a front flank of the pad body joined to the pad lid for discharging the gel to the outside. In this instance, it is preferable that the pad body coupled with the pad lid has a protruding inlet, which is formed at the front flank thereof and has a male screw hole, and the pad lid coupled with the pad body has a coupling hole, which is formed at the front flank thereof and has a female screw hole screw-coupled with the male screw hole of the protruding inlet, so that the protruding inlet and the coupling hole are screw-coupled with each other to thereby join the pad body and the pad lid together. Alternatively, the gel pad cap comprises a pad body and a pad lid formed integrally in such a fashion that the pad body which is sealed by the pad lid is opened when a user takes off and separates the pad lid.

### ADVANTAGEOUS EFFECTS

Because the gel pad cap is forcedly detachably fit to the inlet of the condom to seal the condom and contains the gel therein, even though a sex medicine for men is applied to the inner surface of the condom, the condom according to the present invention can protect the sex medicine for men from external environment and prevent that the sex medicine for men is mixed with the gel. Therefore, the condom according to the present invention makes the sex medicine for men and the gel show their own functions. Moreover, because the gel is kept and carried in safety together with the condom, the user can conveniently use the gel while using the condom even though the user does not separately carry a gel container.

### DESCRIPTION OF DRAWINGS

FIG. 1 is a view showing a condom according to the present invention.

FIG. 2 is a view showing a gel pad (20) of FIG. 1.

FIG. 3 is a view showing various forms of the condom according to the present invention.

### MODE FOR INVENTION

Reference will be now made in detail to the preferred embodiment of the present invention with reference to the attached drawings. The present invention is not restricted to the embodiments of the present invention but can be embodied in other various forms. The following embodiment of the present invention is provided in order to completely disclose the present invention and perfectly let those skilled in the art understand the contents and the scope of the invention, and hence, it will be understood by those of ordinary skill in the art that various changes in form and details may be made therein without departing from the spirit and scope of the present invention. In the drawings, the same components have the same reference numerals having the same functions, and hence, repeated descriptions of the same components having the same functions or structures will be omitted.

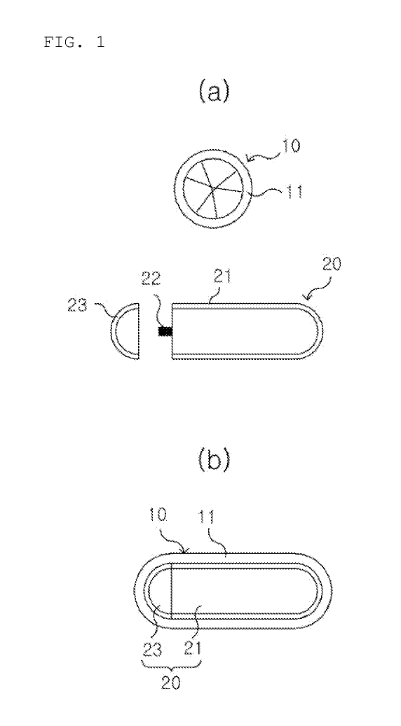

FIG. 1 is a view showing a condom according to the present invention, wherein FIG. 1a is an exploded view showing an exploded state of the condom, and FIG. 1b is a view showing an assembled state of the condom according to the present invention. As shown in FIG. 1, the condom according to the present invention includes a gel pad cap 20 fit to an inlet ring 11 in a rolled state of the condom 10 to stop an inlet opening of the condom.

The gel pad cap 20 includes a pad body 21 and a pad lid 23 is generally in the same form as the inlet ring 11 of the condom and is in a flat plate form to stop the inlet ring 11 of the condom in a state where the pad body 21 and the pad lid 23 are coupled together. Accordingly, when the gel pad cap 20 is fit to the condom 10, it generally has a thin plate shape.

A gel is contained inside the pad body 21, and a protruding inlet 22 to which the pad lid 22 is joined is formed at the front side of the pad body 21 onto which the pad lid 23 abuts and is coupled.

As shown in FIG. 1b, while a user carries the condom 10 to which the gel pad cap 20 is fit, if he wants to use the condom, he separates the gel pad cap 20 from the condom 10 and wears the condom 10, and then, opens the pad lid 23 of the gel pad cap 20 and applies the gel to the outer surface of the condom 10, so that the user can enjoy a desirable sexuality.

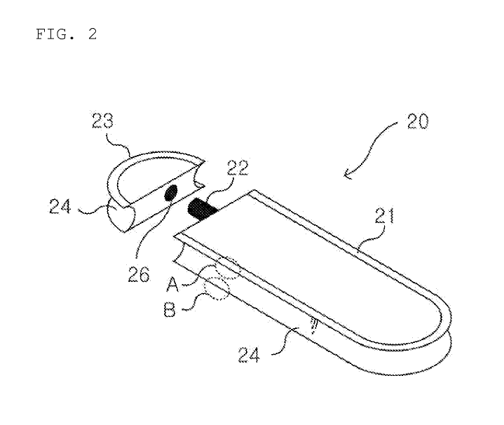

FIG. 2 is a view showing the gel pad cap 20 of FIG. 1. As shown in FIG. 1, the gel pad cap 20 takes the plate form in the state where the pad body 21 and the pad lid 23 are joined together, and the rim of the outer face of the gel pad cap 20 has the same shape as the inlet ring 11 of the condom 10 so as to be closely fit to the inlet ring 11 of the condom.

In order to prevent that the inlet ring 11 of the condom is easily separated when the inlet ring 11 of the condom is fit aside in the state where the pad body 21 and the pad lid 23 are joined together, the gel pad cap 20 has a ring insertion groove 24 formed on a flank of the outer face thereof. The inlet ring 11 of the condom gets in close contact with the ring insertion groove 24 of the gel pad cap 20 because an upper end (A) and a lower end (B) of the ring insertion groove 24 protrude aside and press the inlet ring 11 of the condom from the top and the bottom even though a size of the circumference of the ring insertion groove 24 is equal to that of the inlet ring 11.

The pad lid 23 and the pad body 21 are joined and coupled together, and the pad body 21 coupled with the pad lid 23 has a protruding inlet 22 formed at the front flank thereof and having a male screw hole, and the pad lid 23 coupled with the pad body 21 has a coupling hole 26 formed at the front flank thereof and having a female screw hole screw-coupled with the male screw hole of the protruding inlet 22. The pad lid 23 and the pad body 21 may be joined together not by screw-coupling but by other coupling method. For instance, the protruding inlet 22 and the coupling hole 26 may have not the screw hole but stepped portions, so that the protruding inlet 22 and the coupling hole 26 may be joined together in a snap-joint type when the stepped portions are caught to each other.

The pad lid 23 and the pad body 21 may be formed integrally. In this instance, the pad body 21 is sealed by the pad lid 23, but when the user takes off and separates the pad lid 23, the pad body 21 is opened.

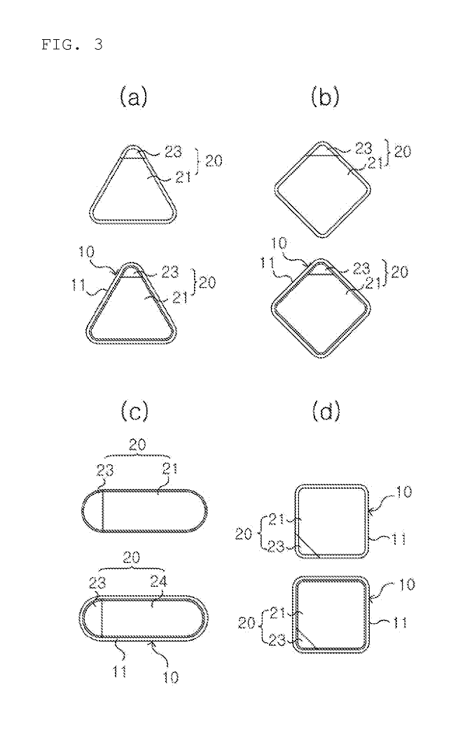

FIG. 3 shows various forms of the condom according to the present invention. As shown in FIG. 3, because the inlet ring 11 of the condom has a circular shape, in order to prevent a separation of the inlet ring 11 from the gel pad cap 20 and tightly join the gel pad cap 20 and the inlet ring 11 when the inlet ring 11 of the condom presses the outer surface of the gel pad cap 20 by a compressive force through an elastic force of the inlet ring 11, preferably, the gel pad cap 20 has not the circular shape but a different shape, for instance, a triangular shape (see FIG. 3a), a diamond shape (see FIG. 3b), an oval shape (see FIG. 3c), a rectangular shape (see FIG. 3d), or other polygonal shape. It is not good that the gel pad cap 20 has a concave portion, for instance, has a star shape, because the gel pad cap 20 cannot be closely joined and coupled with the inlet ring 11 of the condom.

Of course, the gel pad cap 20 may have a circular shape, and in this case, as shown in FIG. 2, it is preferable that the size of the circumference of the ring insertion groove 24 is a little larger than the inlet ring 11 of the condom.

Because the gel pad cap 20 is forcedly detachably fit to the inlet of the condom 10 to seal the condom and contains the gel therein, even though a sex medicine for men is applied to the inner surface of the condom 10, the condom according to the present invention can protect the sex medicine for men from external environment and prevent that the sex medicine for men is mixed with the gel. Therefore, the condom according to the present invention makes the sex medicine for men and the gel show their own functions. Moreover, because the gel is kept and carried in safety together with the condom 10, the user can conveniently use the gel while using the condom even though the user does not separately carry a gel container.

### Explanation Of Essential Reference Numerals In Drawings

10: condom
11: inlet ring of condom
20: gel pad cap
21: pad body
22: protruding inlet
23: pad lid
26: coupling hole

1. A condom comprising
a gel pad cap detachably fit to an inlet ring of the condom to thereby stop an inlet of the condom in a state where the condom is rolled, the gel pad cap containing a gel therein.

2. The condom according to claim 1, wherein the gel pad cap comprises a ring insertion groove formed on a flank of the outer face thereof so that the inlet ring of the condom is closely fit to the gel pad cap.

3. The condom according to claim 2, wherein the gel pad cap and the inlet ring of the condom are closely fit with each other when the inlet ring 11 of the condom presses the outer surface of the gel pad cap 20 by a compressive force through an elastic force of the inlet ring, so that the gel pad cap is detachably fit to the inlet ring of the condom.

4. The condom according to claim 3, wherein in the rolled state of the condom, the inlet ring of the condom is in a circular shape and the gel pad cap is in an oval shape or a polygonal shape.

5. The condom according to claim 3, wherein in the rolled state of the condom, the inlet ring of the condom is in a circular shape and the gel pad cap is also in a circular shape.

6. The condom according to claim 1, wherein the gel pad cap is in a flat plate form.

7. The condom according to claim 1, wherein the gel pad cap comprises a pad body and a pad lid which are separably joined, and the pad body stores a gel therein and has an inlet formed at a front flank of the pad body joined to the pad lid for discharging the gel to the outside.

8. The condom according to claim 7, wherein the pad body coupled with the pad lid has a protruding inlet, which is formed at the front flank thereof and has a male screw hole, and the pad lid coupled with the pad body has a coupling hole, which is formed at the front flank thereof and has a female screw hole screw-coupled with the male screw hole of the protruding inlet, so that the protruding inlet and the coupling hole are screw-coupled with each other to thereby join the pad body and the pad lid together.

9. The condom according to claim 1, wherein the gel pad cap comprises a pad body and a pad lid formed integrally in such a fashion that the pad body which is sealed by the pad lid is opened when a user takes off and separates the pad lid.
